(19) United States
(12) Plant Patent Application Publication
(10) Pub. No.: US 2019/0069458 P1
(43) Pub. Date: Feb. 28, 2019

(54) STRAWBERRY PLANT NAMED 'OMAHA'

(50) ;

(71) Applicant: Sweet Darling Sales, Inc., Aptos, CA (US)

(72) Inventor: John LARSE, Watsonville, CA (US)

(21) Appl. No.: 15/998,933

(22) Filed: Aug. 23, 2018

Related U.S. Application Data
(60) Provisional application No. 62/550,183, filed on Aug. 25, 2017.

(51) Int. Cl. A01H 6/74

(52) U.S. Cl. PLT/209

(57) ABSTRACT

The present invention provides new and distinct strawberry plant designated as ‘Omaha’ (a.k.a. ‘109393’).

### CROSS-REFERENCE TO RELATED APPLICATION

The present application claims priority to, and the benefit of U.S. Provisional Patent Application No. 62/550,183, filed on Aug. 25, 2017, which is herein incorporated by reference in its entirety for all purposes.

Latin name of the genus and species: Fragaria x ananassa.

Varietal denominations: ‘Omaha’ (a.k.a. ‘109393’).

### BACKGROUND OF THE INVENTION

The present invention relates to new and distinct strawberry plant designated as ‘Omaha’ (a.k.a. ‘109393’).

‘Omaha’ (a.k.a. ‘109393’) is the result of a controlled-cross between a female parent cultivar designated ‘Taia’ (U.S. Plant Pat. No. 28,724) and a male parent cultivar designated ‘Ginza’ (U.S. Plant Pat. No. 23,934) made by the Inventor and was first fruited in Watsonville, Calif. growing fields. Following selection and during testing, the plant was originally designated ‘109393’ and subsequently named ‘Omaha’. ‘Omaha’ is a day-neutral plant.

The new strawberry plant was asexually reproduced via runners (stolons) by the inventor at Watsonville, Calif. Asexual propagules from the original source have been tested in Watsonville growing fields and to a limited extent, grower fields in high elevation. The properties of these plant were found to be transmissible by such asexual reproduction. These plants are stable and reproduces true to type in successive generations of asexual reproduction.

### BRIEF SUMMARY OF THE INVENTION

This invention relates to new and distinctive strawberry plant designated as ‘Omaha’ (a.k.a. ‘109393’). The plant is primarily adapted to the climate and growing conditions of the central coast of California. This region provides the necessary temperatures required for it to produce a strong vigorous plant and to remain in fruit production from March through October. The nearby Pacific Ocean provides the needed humidity and moderate day temperatures and evening chilling to maintain fruit quality for the production months.

The following traits and photographs in combination distinguish strawberry plant ‘Omaha’ from known strawberry plants. In addition, these plants were confirmed, or will be confirmed to be a unique strawberry germplasm using Short Sequence Repeats (SSRs). Plants for the botanical measurements in the present application were grown as annuals. Any color references are made to The Royal Horticultural Society Colour Chart, 1995 Edition, except where general terms of ordinary dictionary significance are used.

When ‘Omaha’ is compared to the male parent, ‘Omaha’ is a smaller plant. Additionally, ‘Omaha’ presents fruit with a stronger red color and with a longer fruit shape than the male parent. When ‘Omaha’ is compared to the female parent, ‘Omaha’ presents a higher strawberry pH than the strawberries of the female parent. Additionally, ‘Omaha’ is less dense with foliage and presents fruit with a longer fruit shape than the female parent.

### DESCRIPTION OF THE DRAWINGS

The accompanying color photographs depict various characteristics of the cultivars as nearly true as possible to make color reproductions.

FIG. 1A shows ‘Omaha’ plant about 6-month old.

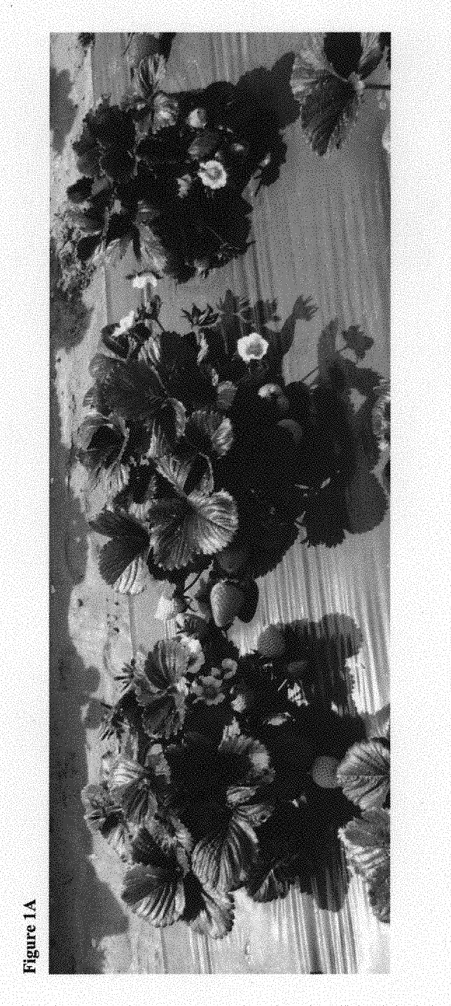

FIG. 1B shows ripe and near-ripe fruits of ‘Omaha’ about 8-month old.

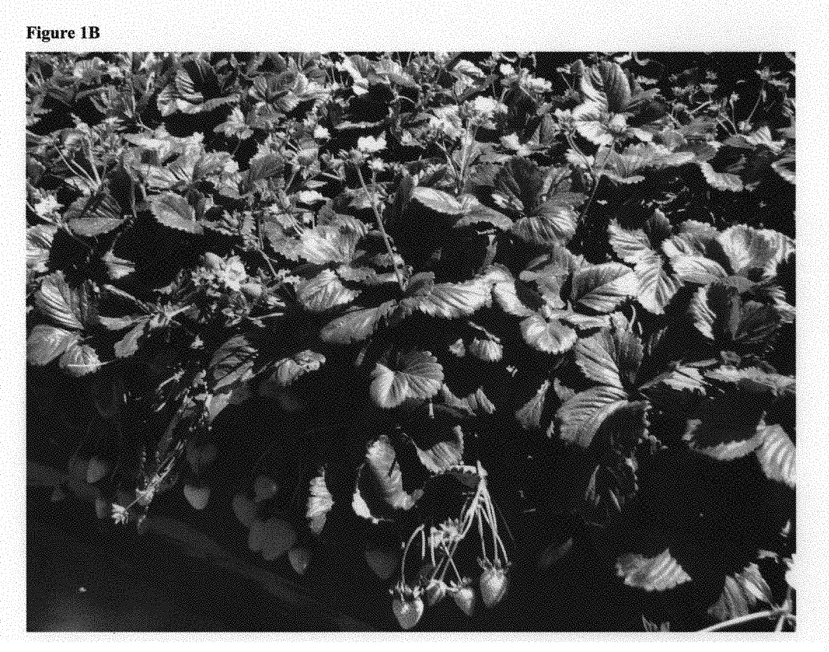

FIG. 1C shows flowers of ‘Omaha’.

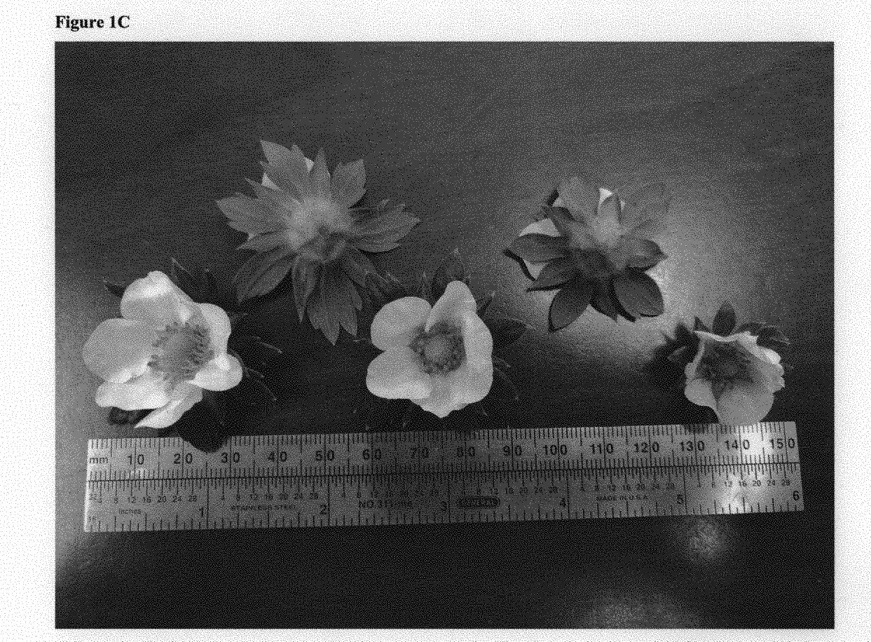

FIG. 1D shows fruits of ‘Omaha’.

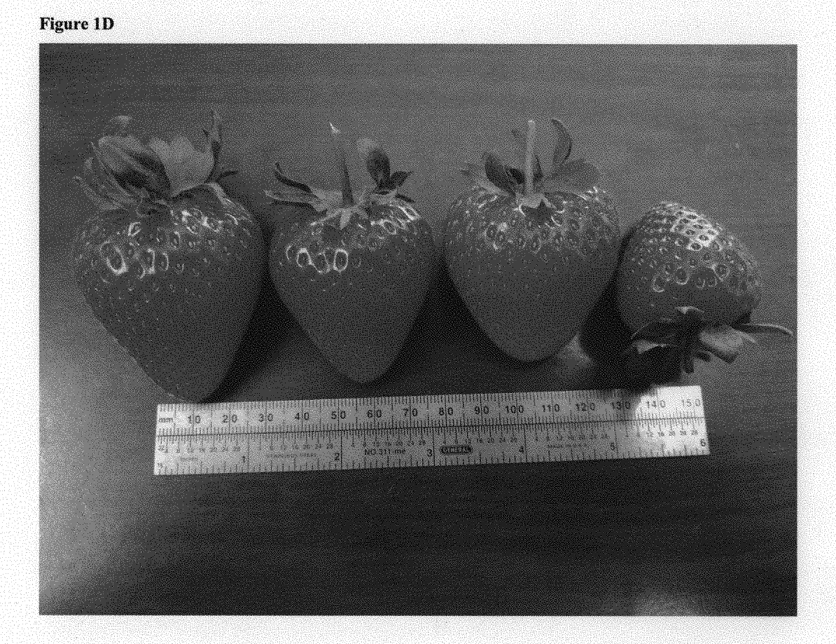

FIG. 1E shows cut fruits of ‘Omaha’.

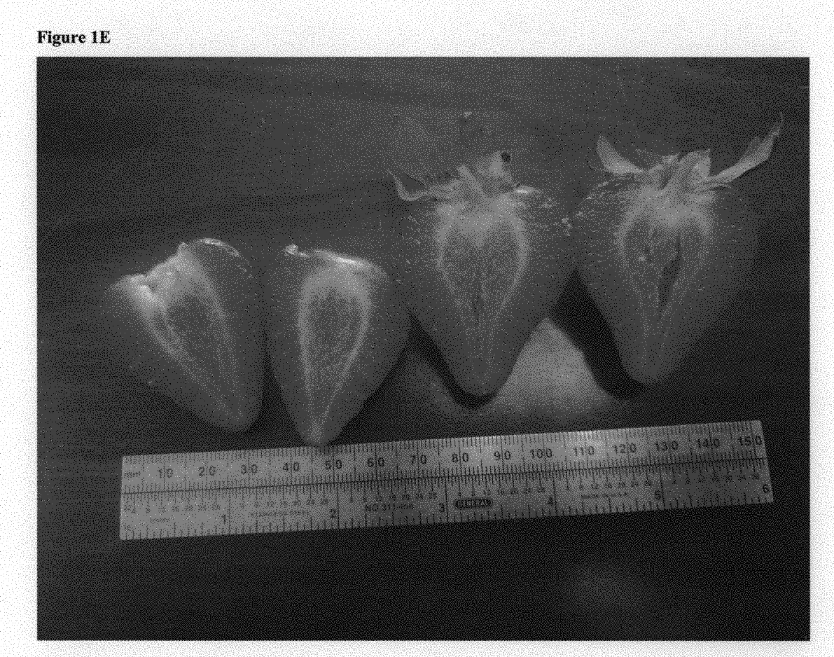

FIG. 1F shows upper leaf of ‘Omaha’.

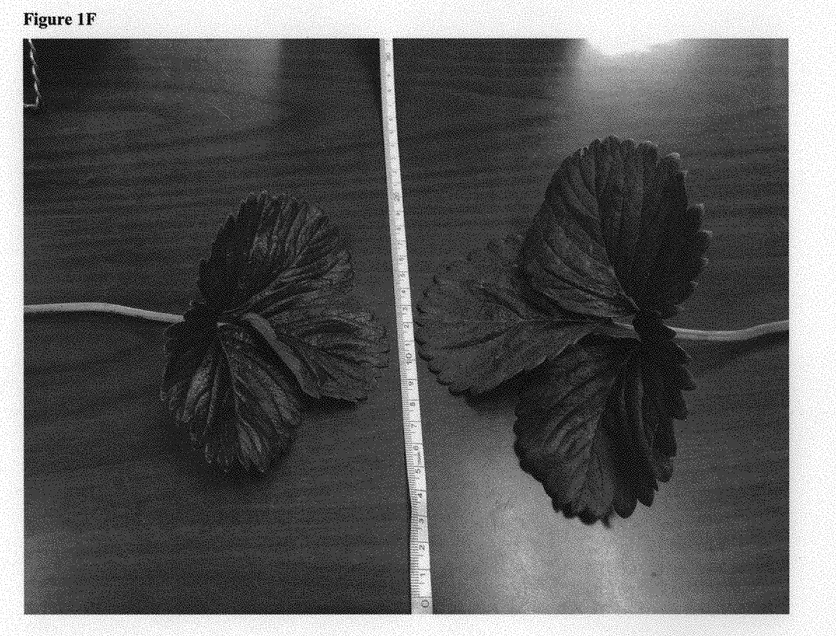

FIG. 1G shows lower leaf of ‘Omaha’.

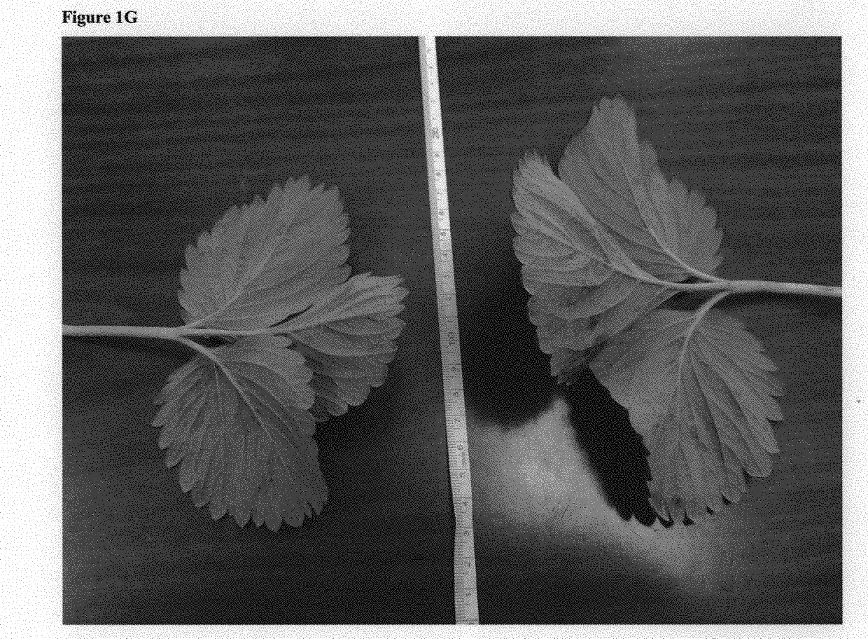

FIG. 1H shows petiole of ‘Omaha’.

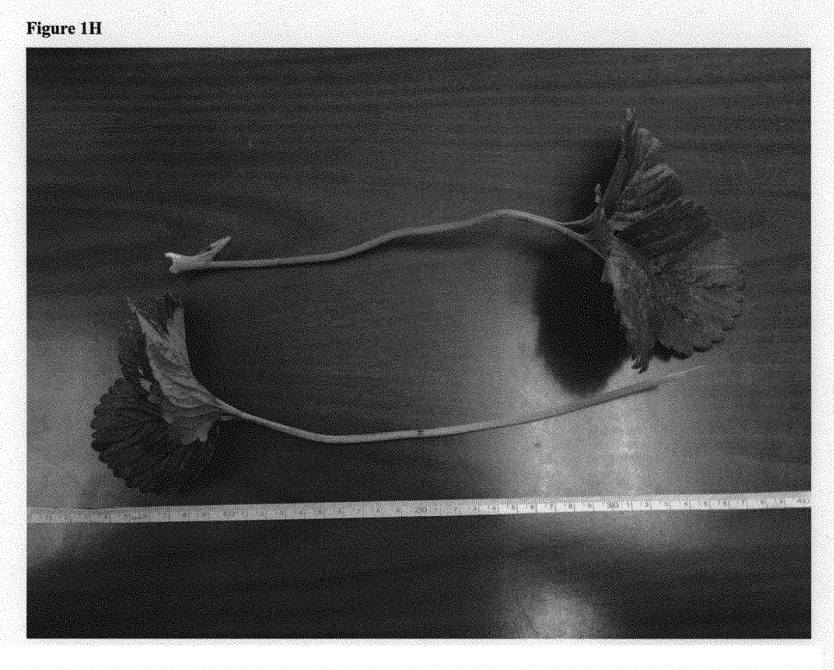

### DETAILED DESCRIPTION OF THE INVENTION

### ‘Omaha’ (a.k.a. ‘109393’)

The following traits in combination distinguish strawberry variety ‘Omaha’ from the known strawberry varieties. Plants for the botanical measurements in the present application were grown as annuals. In the following description, color references are made to The Royal Horticultural Society Colour Chart, 1995 Edition, except where general terms of ordinary dictionary significance are used.

‘Omaha’ has not been observed under all possible environmental conditions, and the phenotype may vary significantly with variations in environment. The following observations, measurements, and comparisons describe this plant as grown under normal conditions in Watsonville, Calif. unless otherwise noted.

TABLE 1
‘Omaha’

| CharType | Characteristic | ‘Omaha’ |
|---|---|---|
| General | Plant Habit | annual |
| | Plant Growth Habit | semi-upright |
| | Day length | Day Neutral |
| | Plant Height (cm) | 28 |
| | Plant Width (cm) | 33 |
| | Density of foliage | medium |
| | Plant vigor | moderate to high |
| Leaf | Terminal leaflet width (mm) | 8.9 |
| | Terminal leaflet length (mm) | 7.4 |
| | No. teeth/terminal leaflet: | 23 |
| | Shape of the terminal leaflet base | acute to obtuse |
| | Shape of terminal leaflet in cross- | striaight to concave |
| | section | |
| | Margin description of the terminal | scrrate to crenate |
| | Color of upper side of leaves | 137A |
| | Color of lower side of leaves | 137C |
| | Leaf blistering | weak |
| | Leaf glossiness | medium |
| Limbs | Petiole length (cm) | 20 to 25 |
| | Petiole diameter (mm) | 3.28 |
| | Petiole color | 145A |
| | Petiolule length (mm) | 1 |
| | Petiolule diameter (mm) | 2.28 |
| | Stipule length (cm) | 2.8 |
| | Stipule width (cm) | 1.1 |
| | Stipule anthocyanin | absent |
| | Stipule color (color code) | 145A |
| | Pedicel color (color code) | 145A |
| | attitude of hairs on | outwards |
| | petiole and pedicel | |
| Inflorescence | Inflorescence position relative to | above |
| | foliage | |
| | Flower arrangement of petals | touching |
| | Petal length (cm) | 1.4 |
| | Petal width (cm) | 1.4 |
| | Petal number per flower | 5 to 6 |
| | Upper Petal color | 155C |
| | Lower Petal color | 155C |
| | Peduncle size | medium |
| | Calyx diameter (cm) | 4 |
| | Corolla diameter (cm) | 2.75 |
| | Sepal length (cm) | 1.1 to 2.1 |
| | Sepal width (cm) | 0.8 to 1.1 |
| | Time of flowering | March |
| | (50% of plants in bloom) | |
| | Shape of stigma | capitate |
| | Color of stigma | 6A |
| | Length of style (mm) | 2 |
| | Color of style | 6A |
| | Color of the ovary | 145A |
| | Length of the stamens (mm) | 3 to 5.5 |
| | Number of stamen | 24 to 33 |
| | Shape of anther | dorsifixed |
| | Size of anther | small |
| | Color of anther | 12A |
| | Amount of pollen | medium |
| | Color of pollen | 4A |
| | Color of filament | 149D |
| | Length of filament (mm) | 2.5 to 5 |
| | Number of flowers per truss | 3 to 6 |
| Stolon | Stolon number | 2 to 5 |
| | Stolon anthocyanin | 181A |
| | Widest diameter of stolon | 3.83 |
| | At leaf attachment (mm) | |
| | Stolon color | 145 |
| Fruit | Number of fruit per truss | 3 to 4 |
| | Fruit length (cm) | 40.4 |
| | Fruit width (cm) | 30.7 |
| | Fruit skin color | 45A |
| | Fruit flesh color excluding core | 44A |
| | Fruit core color | 41A |
| | Fruit weight (g) | 23.6 |
| | Predominant fruit shape | long conic |
| | Shape difference between | No shape difference |
| | primary & secondary fruits | |
| | Width of band without of achenes | narrow |
| | Fruit glossiness | medium to strong |
| | Position of achenes | below surface |
| | Achene color | 145A |
| | Achenes per fruit | 273 |
| | Achene weight (g) | 0.19 |
| | Position of calyx | inserted |
| | level of adherence of calyx | medium |
| | Color of calyx | 137A |
| | Firmness of flesh | medium to firm |
| | Evenness of flesh color | even |
| | Sweetness (brix) | 6 to 7 |
| | pH | 3.51 |
| | Yield (g per plant per season) | 2758 |

‘Albion’ (U.S. Plant Pat. No. 16,228) owned by University of California is a commercial strawberry variety that is similar to, but distinguished from ‘Omaha’. The fruit yield of ‘Omaha’ exceeds the yield of ‘Albion’ in multiple annual testing cycles performed at the breeders test location in Watsonville Calif. ‘Omaha’ produces abundant quantities of stolon that must be cut by hand labor in the fruiting field. ‘Omaha’ does not produce large quantities of stolon (runners) during the fruiting period after propagation in California nurseries and Watsonville fruiting field, which saves labor of about $1,500 per acre because stolon must be removed in order to effect efficient harvest and continued flowering.

Both of the parents of ‘Omaha’ produce fruit that is lighter in redness coloration than ‘Omaha’. One of the parents of ‘Omaha’, a strawberry plant variety named ‘Ginza’ (US Plant Pat. No. 23,934) has been observed to have foliage of a lighter green coloration than ‘Omaha’. The foliage and plant structure of ‘Ginza’ is more dense than that of ‘Omaha’. The other parent of ‘Omaha’ is a strawberry plant variety named ‘Taia’ (U.S. Plant Pat. No. 28,724). The foliage and plant structure of ‘Taia’ is somewhat more dense than that of ‘Omaha’. The foliage and plant structure of ‘Omaha’ allows better fruit harvest visibility and the canopy can be described as more open than the canopy observed for of each of it's parents. ‘Omaha’ demonstrated better shelf life, less susceptibility to bruising discoloration and the skin of the fruit of ‘Omaha’ was observed to better resist abrasion compared to each of it's parents in testing done over multiple weeks of annual fruiting seasons.

1. A new and distinct cultivar of strawberry plant named ‘Omaha’ substantially as shown and described herein.
